(19) United States
(12) Patent Application Publication
(10) Pub. No.: US 2002/0022746 A1
(43) Pub. Date: Feb. 21, 2002

(54) Method for the crystallization of iopamidol

(76) Inventors: Jin Kyu Park, Seoul (KR); Kyung Seok Choi, Icheon City (KR); Dong Yub Lee, Cheongju City (KR); Chang Hun Song, Cheongju City (KR); Dong Jun Lee, Jecheon City (KR)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/852,140

(22) Filed: May 9, 2001

Related U.S. Application Data
(63) Continuation of application No. PCT/KR99/00181, filed on Apr. 16, 1999.

(30) Foreign Application Priority Data
Nov. 9, 1998 (KR) 1998-47785

(51) Int. Cl. C07C 231/24

(52) U.S. Cl. 564/153

(57) ABSTRACT

Disclosed is the crystallization of Iopamidol using a mixture of an alcohol and water as a crystallization solvent. The ratios of water:Iopamidol from 1:10 to 1:1 (v/w) and alcohol:Iopamidol from 1:1 to 1:10 (v/w) allow Iopamidol to be crystallized to an anhydrous pure form with industrially valuable yields without producing monohydrates and pentahydrates.

### TECHNICAL FIELD

The present invention relates to a novel method for crystallizing Iopamidol to an anhydrous form, which is generally used as an X-ray contrast medium. More particularly, the present invention is concerned with a method for purifying Iopamidol to pharmaceutically acceptable purity, by which anhydrous Iopamidol can be produced at industrial yields.

### BACKGROUND ART

Crystalline forms of Iopamidol exist as an anhydrous, monohydrated or pentahydrated state. Of them, anhydrous Iopamidol is pharmaceutically acceptable and thus, is now produced industrially. Techniques with which industrially beneficial Iopamidol can be obtained are found in U.S. Pat. Nos. 5,571,941 and 5,689,002.

U.S. Pat. No. 5,571,941 discloses a method for purifying Iopamidol, teaching that use of butanol, such as n-butanol, sec-butanol, isobutanol or t-butanol, as a crystallization solvent allows pharmaceutically pure Iopamidol with industrially acceptable yields. On the other hand, U.S. Pat. No. 5,689,002 discloses a method for crystallizing Iopamidol, in which water is used as a crystallization solvent to yield anhydrous pure Iopamidol which meets the pharmacopeia standards.

### DISCLOSURE OF THE INVENTION

The intensive and thorough research on the crystallization of Iopamidol, repeated by the present inventors, resulted in the surprising finding that a mixture of water and alcohol allows Iopamidol to be crystallized into an anhydrous pure form without producing monohydrates and pentahydrates.

As far as the present invention is concerned, Iopamidol, whether it exists as a monohydrate or a pentahydrate, can be crystallized into highly pure, anhydrous forms with industrially available yields.

Therefore, it is an object of the present invention to provide a method for the crystallization of Iopamidol, by which anhydrous Iopamidol with high purity can be industrially produced.

It is another object of the present invention to provide a method for purifying Iopamidol to pharmaceutically acceptable purity.

These and other objects can be attained in a method for crystalizing Iopamidol, comprising the steps of dissolving Iopamidol in deionized water, distilling a part of the water off under a vacuum condition, adding alcohol to the aqueous solution, subjecting the solution to reflux for 3-4 hours to give white precipitates, filtering the precipitates, and drying the precipitates to yield pure anhydrous Iopamidol.

Particularly preferable is that the deionized water is used at an amount of water:Iopamidol from 1:10 to 1:1 (v/w) and the alcohol is used at an amount of alcohol:Iopamidol from 1:1 to 1:10 (v/w).

Further preferable is that the alcohol is selected from the group consisting of ethanol and propanol.

### BEST MODES FOR CARRYING OUT THE INVENTION

To be useful for injection, Iopamidol, which is used as an X-ray contrast medium, must exist as an anhydrous white crystalline form, but not as monohydrates or pentahydrates, and exhibit a solubility of 80% (w/v) in water. In addition, for industrial application, anhydrous Iopamidol must be produced with high purity at high yields while avoiding residual solvents which may cause an environmental problem.

In accordance with the present invention, a mixture of alcohol and water is used as a crystallization solvent for Iopamidol. The crystallization of the present invention starts by dissolving Iopamidol, whether existing as a monohydrate or a pentahydrate, in deionized water. Under a vacuum condition, this aqueous suspension is concentrated to distill off a part of the deionized water. The concentrated mixture is added with alcohol and brought to reflux for a predetermined period of time. During the reflux, Iopamidol begins to precipitate as white crystals. These precipitates are filtered off and dried.

Useful in the present invention is ethanol or propanol. This alcohol is used at an amount of alcohol:Iopamidol from 1:1 to 1:10 (v/w). As for water, it is used at an amount of water:Iopamidol from 1:10 to 1:1 (v/w).

With reference to FIG. 1, there is a graph showing the solubility of Iopamidol plotted against reflux time when a ratio of Iopamidol:water:ethanol 4:1:8 (w/v/v) is used in accordance with the method of the present invention. As shown in this graph, the reflux for 4 hours or greater allows the crystallized Iopamidol to have a solubility of 80% or greater. This indicates that, when the reflux continues to be performed for 4 hours, only pure anhydrous Iopamidol exists. Because Iopamidol monohydrates or pentahydrates are of about 50% solubility, the method of the present invention can convert Iopamidol monohydrates or pentahydrates into Iopamidol anhydrides.

A better understanding of the present invention may be obtained in light of the following examples which are set forth to illustrate, but are not to be construed to limit the present invention.

### EXAMPLE I

10 kg of Iopamidol was dissolved in 10 liters of deionized water, followed by vacuum distilling 8 liters of the water. After being added with 5 liters of ethanol, the suspension was subjected to reflux for 4 hours. During the reflux, Iopamidol began to precipitate as white crystals which were, then, filtered off. After drying at 60° C. for 4 hours in vacuo, 7.5 kg of pure anhydrous Iopamidol was obtained. Yield: 75%.

### EXAMPLE II

10 kg of Iopamidol was dissolved in 10 liters of deionized water, followed by vacuum distilling 7.5 liters of the water. After being added with 20 liters of ethanol, the suspension was subjected to reflux for 4 hours. During the reflux, Iopamidol began to precipitate as white crystals which were, then, filtered off. After drying at 60° C. for 4 hours in vacuo, 9.3 kg of pure anhydrous Iopamidol was obtained. Yield: 93%.

### EXAMPLE III

10 kg of Iopamidol was dissolved in 10 liters of deionized water, followed by vacuum distilling 8 liters of the water. After being added with 20 liters of propanol, the suspension was subjected to reflux for 4 hours. During the reflux, Iopamidol began to precipitate as white crystals which were, then, filtered off. After drying at 60° C. for 4 hours in vacuo, 9.41 kg of pure anhydrous Iopamidol was obtained. Yield : 94.1%.

### EXAMPLE IV

10 kg of Iopamidol was dissolved in 10 liters of deionized water, followed by vacuum distilling 8 liters of the water. After being added with 30 liters of ethanol, the suspension was subjected to reflux for 4 hours. During the reflux, Iopamidol began to precipitate as white crystals which were, then, filtered off. After drying at 60° C. for 4 hours in vacuo, 9.0 kg of pure anhydrous Iopamidol was obtained. Yield: 90%.

### EXAMPLE V

10 kg of Iopamidol was dissolved in 10 liters of deionized water, followed by vacuum distilling 7.5 liters of the water. After being added with 20 liters of propanol, the suspension was subjected to reflux for 4 hours. During the reflux, Iopamidol began to precipitate as white crystals which were, then, filtered off. After drying at 60° C. for 4 hours in vacuo, 9.4 kg of pure anhydrous Iopamidol was obtained. Yield: 94%.

### TEST EXAMPLE I

In order to determine proper reflux time, the same procedures of the above examples were repeated, except that the reflux was performed for 1, 2, 3, 4 and 5 hours per procedure round. The solubility of the Iopamidol obtained was measured by dissolving 80 g of the Iopamidol in 100 ml of water. The results are given in Table 1, below.

TABLE 1
Solubility of Iopamidol according to Reflux Times

Reflux Times (hour)

| Examples | 1 | 2 | 3 | 4 | 5 |
|---|---|---|---|---|---|
| Example I | 50 | 63 | 98 | 100 | 100 |
| Example II | 51 | 61 | 99 | 100 | 100 |
| Example III | 53 | 62 | 98 | 100 | 100 |
| Example IV | 51 | 63 | 99 | 100 | 100 |
| Example V | 52 | 62 | 100 | 100 | 100 |

### TEST EXAMPLE II

The purity of the anhydrous Iopamidol obtained in the above examples was measured using high performance liquid chromatography (HPLC). The results are given in Table 2, below.

TABLE 2
Purity of Iopamidol

Examples

| | I | II | III | IV | V |
|---|---|---|---|---|---|
| Purity (Area %) | 99.8 | 99.8 | 99.5 | 99.9 | 99.5 |

### BRIEF DESCRIPTION OF THE DRAWING

FIG. 1 is a graph showing the solubility change of the Iopamidol with reflux time.

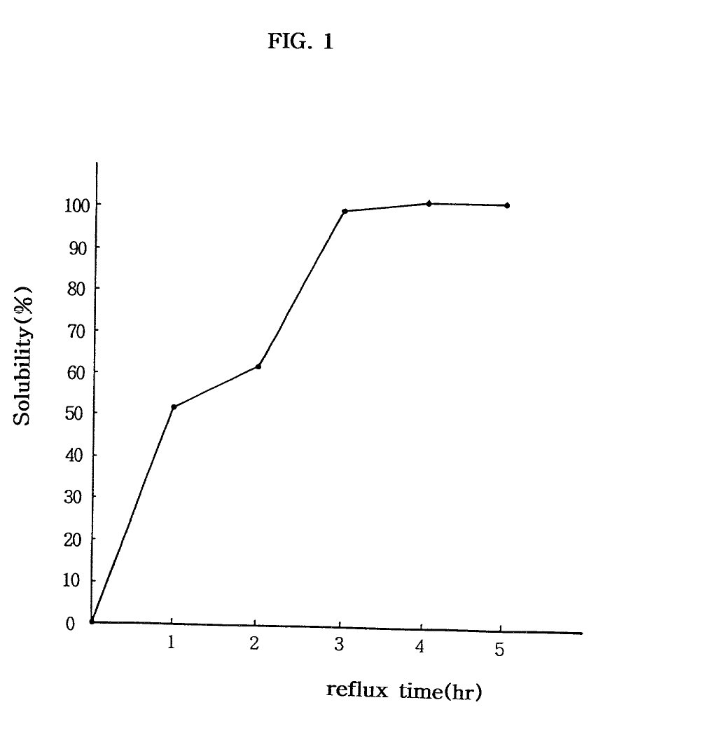

### INDUSTRIAL APPLICABILITY

As described hereinbefore, the crystalizing method of Iopamidol, according to the present invention, does not need active carbon for decolorization, in contrast to the conventional crystalizing technique from water, and nor causes harm to the human body owing to residual solvents which may be problematic in the conventional technique using butanol as a crystallization solvent. In addition, the method of the present invention allows anhydrous Iopamidol 99.5% or higher purity, with a yield of 90%, so that it can be industrially applied for the mass production of Iopamidol.

The present invention has been described in an illustrative manner, and it is to be understood the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

What is claimed is:

1. A method of crystallizing iopamidol, comprising:
providing an iopamidol solution comprising a crystallizing solvent and iopamidol dissolved therein;
adding an alcohol to the iopamidol solution; and
evaporating the crystallizing solvent from the iopamidol solution so as to precipitate solid iopamidol.

2. The method of claim 1, wherein the providing the iopamidol solution comprises dissolving iopamidol in the crystallizing solvent.

3. The method of claim 2, wherein in the dissolving excessive iopamidol forms a suspension of the iopamidol in the iopamidol solution.

4. The method of claim 1, wherein the providing the iopamidol solution comprises adjusting concentration of the iopamidol solution.

5. The method of claim 4, wherein the adjusting concentration comprises evaporating the crystallizing solvent before adding the alcohol.

6. The method of claim 5, wherein the evaporating comprises distilling the iopamidol solution under a negative pressure.

7. The method of claim 1, wherein the crystallizing solvent comprises water.

8. The method of claim 7, wherein the water is deionized water.

9. The method of claim 1, wherein the alcohol comprises at least one of ethanol and propanol.

10. The method of claim 1, wherein the evaporating comprises refulxing the iopamidol solution.

11. The method of claim 1, wherein the evaporating continues from about 3 to about 4 hours.

12. The method of claim 1, wherein a ratio of the crystallizing solvent to iopamidol ranges from about 0.1 to about 1 (v/w).

13. The method of claim 1, wherein a ratio of the alcohol to iopamidol ranges from about 1 to about 10 (v/w).

14. The method of claim 1, wherein the method is performed in an industrial scale.

15. The method of claim 1, wherein the solid iopamidol comprises an anhydrous form of iopamidol.

16. A method of preparing anhydrous form of iopamidol, the method comprising the crystallization method of claim 1, wherein the providing an iopamidol solution comprises dissolving in a crystallizing solvent iopamidol comprising hydrate forms thereof.

17. A method of isolating iopamidol from a mixture including iopamidol, the method comprising the crystallization method of claim 1, wherein the precipitated solid iopamidol is subject to a post-crystallization treatment.

18. The method of claim 17, wherein the post-crystallization treatment comprises separating the solid iopamidol from liquid.

19. The method of claim 17, wherein the post-crystallization treatment comprises drying the solid iopamidol.

20. The method of claim 17, wherein the solid iopamidol comprises an anhydrous form of iopamidol.
